(19) United States
(12) Patent Application Publication
(10) Pub. No.: US 2023/0226240 A1
(43) Pub. Date: Jul. 20, 2023

(54) ROTATABLE WAX-MELTING FRAGRANCE LAMP

(71) Applicant: SHENZHEN XIAOYUAN IMPORT & EXPORT LTD, Shenzhen (CN)

(72) Inventor: XIAOYUAN MA, Shenzhen (CN)

(21) Appl. No.: 17/578,544

(22) Filed: Jan. 19, 2022

(51) Int. Cl. A61L 9/03; H05B 3/06; H05B 1/02; F21V 33/00

(52) U.S. Cl. CPC A61L 9/03; H05B 3/06; H05B 1/0252; F21V 33/0004; H05B 2203/02; H05B 2203/021; F21Y 2115/10

(57) ABSTRACT

A rotatable wax-melting fragrance lamp comprises a base, a housing, a rotating component, a heating component, a light-emitting component, a control PCB, and a heat-conducting plate. By arranging the rotating component, the whole product can present a rotating effect, which can make the lamp have better lighting effect and decorative effect, and enables the heating component to melt and volatilize wax more uniformly; the heat-conducting plate is detachable, which is convenient for cleaning and replacing the fragrant wax; the PTC heating element or ceramic heating resistor is used in place of open fire heater. The heating of the PTC heating element or the ceramic heating resistor is to realize the heating of the fragrant wax, so that the fragrant wax produces a pleasant fragrance, which spreads around the immediate vicinity. The PTC heating element or ceramic heating resistor requires lower heating power consumption, while the power control is very sensitive.

### TECHNICAL FIELD

The present application relates to the technical field of fragrance equipment, in particular to a rotatable wax-melting fragrance lamp.

### BACKGROUND

Scented candles are a more traditional way to produce fragrance to the living environment. In a quiet night, or need to create a romantic atmosphere, candles are one of the essential embellishments. Sparkling flames and pervasive fragrance bring about warm feelings. Fragrance helps to relax the mind and body. Scented candles can be used in any place such as office and residence.

For the wax-melting fragrance equipment and similar products available on the market, the light-emitting and heating functions are provided by the internal tungsten lamp. When the tungsten filament lamp is powered on, the temperature in the wax-melting fragrance equipment will continue to rise over time, with more and more energy consumed. However, with the continuous rise of the temperature, it also brings many problems, such as, when the users, especially children, touch the surface of the fragrance equipment by accident, he or she will be burned. Other problems include non-uniform heating during wax melting, single lighting effect, etc. The wax-melting fragrance equipment can no longer meet contemporary people's living and consumption habits.

### SUMMARY

The purpose of the present application is to provide a rotatable wax-melting fragrance lamp in view of the above-mentioned technical defects, in order to solve the technical problems existing in the prior art, such as non-uniform heating, single lighting effect and excessively high heating power.

In order to solve the above technical problems, the present application adopts the following technical proposal:

The present application provides a rotatable wax-melting fragrance lamp, which comprises a base and housing arranged on the base, a rotating component is arranged between the base and the housing, a heating component is arranged at the top of the housing, a light-emitting component is arranged at the bottom of the heating component, a control PCB is arranged on the upper side of the base, wherein the rotating component, the heating component and the light-emitting component are configured to electrically couple with the control PCB through wires, and the control PCB is configured to electrically couple with an external power source through a wire.

Further, the rotating component comprises a motor tray, a motor and a turntable. The motor tray is fixed to the upper side of the base, the motor is fixed to the motor tray, and the turntable is fixed to the rotating shaft of the motor.

Further, a circular hoop is fixedly arranged on the outer circumference of the turntable, and the circular hoop can be clipped into the housing.

Further, the light-emitting component comprises LED beads, and the LED beads are fixedly arranged on the bottom of the heating component facing the center of the housing.

Further, the light-emitting component also comprises a rotating interior view ring, which is fixedly arranged on the rotating component inside the housing.

Further, the light-emitting component comprises a heating resistor tray, a heating resistor and a fixing ring. The heating resistor tray is fixed to the upper opening of the housing through the fixing ring, and the heating resistor is fixedly arranged in the middle of the heating resistor tray.

Further, the heating resistor is a PTC heating element or a ceramic heating resistor.

Further, the rotatable wax-melting fragrance lamp also comprises a heat-conducting plate, which is detachable arranged on the fixing ring and is located above the heating resistor.

Further, the housing is made of glass fiber or transparent plastic.

Compared with the prior art, the present application has beneficial effects as follows: By arranging the rotating component, the whole product can present a rotating effect, which can make the lamp have better lighting effect and decorative effect, and enables the heating component to melt and volatilize wax more uniformly; the PTC heating element or the ceramic heating resistor is used in place of open fire heater, so that the heating power is minimal and power control is very sensitive, making it easy to control temperature; the heat-conducting plate is detachable, which is convenient for cleaning and replacing the fragrant wax;

### BRIEF DESCRIPTION OF DRAWINGS

FIG. 1 is a structural diagram of the rotatable wax-melting fragrance lamp according to an embodiment of the present application;

FIG. 2 is a breakdown structure diagram of the rotatable wax-melting fragrance lamp according to an embodiment of the present application.

The drawing reference signs are explained as follows:

1. Base, 2. Housing, 3. Rotating component, 4. Heating component, 5. Light-emitting component, 6. Control PCB, 7. Heat-conducting plate, 301. Motor tray, 302. Motor, 303. Turntable, 304. Circular hoop, 401. Heating resistor tray, 402. Heating resistor, 403. Fixing ring, 501. LED beads, 502. Rotating interior view ring.

### DETAILED DESCRIPTION

The present application is further described below with reference to the drawings.

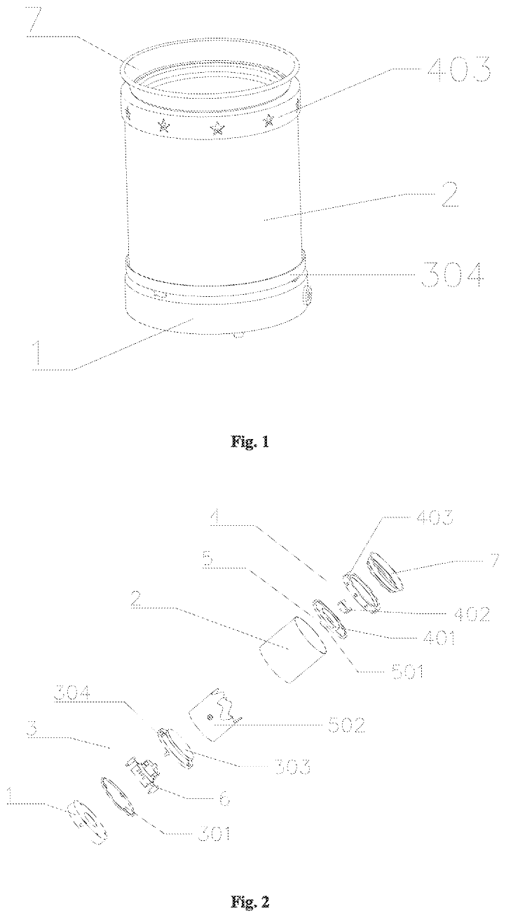

According to FIGS. 1 and 2, in one embodiment, the present application provides a rotatable wax-melting fragrance lamp. The rotatable wax-melting fragrance lamp comprises a base 1 and a housing 2 arranged on the base 1. Importantly, a rotating component 3 is arranged between the base 1 and the housing 2. A heating component 4 is arranged at the top of the housing 2. A light-emitting component 5 is arranged at the bottom of the heating component 4. A control PCB 6 is arranged on the upper side of the base 1. The rotating component 3, the heating component 4 and the light-emitting component 5 are configured to electrically couple with the control PCB 6 through wires, the control PCB 6 is configured to electrically couple with an external power source through a wire, and the current flowing through the control PCB 6 controls the operation of the product. By arranging the rotating component 3, the whole product can present a rotating effect, which can make the lamp have better lighting effect and decorative effect, and enables the heating component 5 to melt and volatilize wax more uniformly.

Specifically, the rotating component 3 comprises a motor tray 301, a motor 302 and a turntable 303, the motor tray 301 is fixed to the upper side of the base 1, the motor 302 is fixed to the motor tray 301, and the turntable 303 is fixed to the rotating shaft of the motor 302. The turntable 303 is mainly driven by the power provide by the motor 302, so that a rotating effect is achieved.

More specifically, a circular hoop 304 is fixedly arranged on the outer circumference of the turntable 303, and the circular hoop 304 can be clipped into the housing 2. This design has a simple structure and is more convenient to assemble.

Specifically, the light-emitting component 5 comprises LED beads 501, and the LED beads 501 are fixedly arranged on the bottom of the heating component 4 facing the center of the housing 2 in order to achieve a lighting effect. According to the actual situation, LED bead with different brightness and different colors can be selected to achieve different lighting effects.

More specifically, the light-emitting component 5 also comprises a rotating interior view ring 502, and the rotating interior view ring 502 is fixedly arranged on the rotating component 3 inside of the housing 1. By creating some shadow effects, different lighting effects, such as flames, jungles, etc. could be achieved.

Specifically, the light-emitting component 4 comprises a heating resistor tray 401, a heating resistor 402 and a fixing ring 403. The heating resistor tray 401 is fixed to the upper opening of the housing 2 through the fixing ring 403, and the heating resistor 402 is fixed in the middle of the heating resistor tray 401 in place of the traditional heat source, so that an open fire is not generated, increasing device safety.

More specifically, the heating resistor 402 is a PTC heating element or a ceramic heating resistor, and the PTC heating element or the ceramic heating resistor is to realizes the heating of the fragrant wax, so that the fragrant wax produces a pleasant fragrance, which spreads around the immediate vicinity. The PTC heating element or ceramic heating resistor requires lower heating power consumption, while power control response is very sensitive, which is convenient for customers to control the temperature.

More specifically, the rotatable wax-melting fragrance lamp further comprises a heat-conducting plate 7, which can detachable arranged on the fixing ring and is located above the heating resistor, and is mainly used to heat the fragrant wax. When it does not need to be replaced or cleaned, it can be removed directly, which makes it more convenient to use.

More specifically, the housing 2 is made of glass fiber or transparent plastic, which is mainly light-transmitting. Of course, it can be designed to have light-transmitting materials with different colors according to the needs of the situation.

In addition to the above, it should be noted that “one embodiment”, “another embodiment”, the “embodiments” and other relevant terms referred to in this specification mean that the specific features, structure or characteristics described in connection with the related embodiment are included in at least one embodiment generally described in this application. The same description that occurs in multiple parts of the specification does not necessarily refer to the same embodiment. Further, when a specific feature, structure or characteristic is described in connection with any embodiment, it is claimed that the implementation of this feature, structure or characteristic in other embodiments also fall within the scope of the present application.

Although the present application has been described herein with reference to a number of explanatory embodiments of the present application, it should be understood that many other modifications and embodiments designed by experts shall fall within the scope of the present application. More specifically, the components and/or layout of the embodiments can be modified and improved in many ways within the scope of the present application, the drawings, and the claims. In addition to modifications and improvements in the components and/or layout of the embodiments, other uses will also be apparent to those skilled in the art.

What is claimed is:

1. A rotatable wax-melting fragrance lamp, comprising a base and a housing arranged on the base, wherein a rotating component is arranged between the base and the housing, a heating component is arranged at the top of the housing, a light-emitting component is arranged at the bottom of the heating component, a control PCB is arranged on the upper side of the base, the rotating component, the heating component and the light-emitting component are configured to electrically couple with the control PCB through wires, and the control PCB is configured to electrically couple with an external power source through a wire.

2. The rotatable wax-melting fragrance lamp as recited in claim 1, wherein the rotating component comprises a motor tray, a motor and a turntable, the motor tray is fixed to the upper side of the base, the motor is fixed on the motor tray, and the turntable is fixed to the rotating shaft of the motor.

3. The rotatable wax-melting fragrance lamp as recited in claim 2, wherein a circular hoop is fixedly arranged on the outer circumference of the turntable, and the circular hoop can be clipped into the housing.

4. The rotatable wax-melting fragrance lamp as recited in claim 1, wherein the light-emitting component comprises LED beads, and the LED beads are fixedly arranged on the bottom of the heating component facing the center of the housing.

5. The rotatable wax-melting fragrance lamp as recited in claim 4, wherein the light-emitting component further comprises a rotating interior view ring, which is fixedly arranged on the rotating component inside the housing.

6. The rotatable wax-melting fragrance lamp as recited in claim 1, wherein the light-emitting component comprises a heating resistor tray, a heating resistor and a fixing ring, the heating resistor tray is fixed to the upper opening of the housing through the fixing ring, and the heating resistor is fixedly arranged in the middle of the heating resistor tray.

7. The rotatable wax-melting fragrance lamp as recited in claim 6, wherein the heating resistor is a PTC heating element or a ceramic heating resistor.

8. The rotatable wax-melting fragrance lamp as recited in claim 6, comprising a heat-conducting plate, which is detachable arranged on the fixing ring and is located above the heating resistor.

9. The rotatable wax-melting fragrance lamp as recited in claim 1, wherein the housing is made of glass fiber or transparent plastic.
